(19) United States
(12) Patent Application Publication
(10) Pub. No.: US 2026/0078093 A1
(43) Pub. Date: Mar. 19, 2026

(54) (4R)-3-(4-FLUORO-2-HYDROXYPHENYL)-4-METHYL-4,5-DIHYDRO-1H-PYRAZOLE-1-CARBOXIMIDAMIDE HYDROCHLORIDE AND DERMAL FORMULATIONS THEREOF

(71) Applicant: IMMUNAHR AB, Lund (SE)

(72) Inventor: Lars PETTERSSON, Lund (SE)

(73) Assignee: IMMUNAHR AB, Lund (SE)

(21) Appl. No.: 19/394,514

(22) Filed: Nov. 19, 2025

Related U.S. Application Data
(63) Continuation-in-part of application No. PCT/EP2024/064241, filed on May 23, 2024.
(63) Continuation-in-part of application No. PCT/EP2024/064242, filed on May 23, 2024.

(30) Foreign Application Priority Data
May 26, 2023 (EP) 23175606.5
May 26, 2023 (EP) 23175612.3

(51) Int. Cl. C07D 231/06; A61K 9/00; A61K 9/06; A61K 31/415; A61K 47/10; C07C 69/76; C07C 225/16; C07D 231/12

(52) U.S. Cl. CPC C07D 231/06; A61K 9/0014; A61K 9/06; A61K 31/415; A61K 47/10; C07C 69/76; C07C 225/16; C07D 231/12

(57) ABSTRACT

The present invention relates to the compound (4R)-3-(4-fluoro-2-hydroxyphenyl)-4-methyl-4,5-dihydro-1H-pyrazole-1-carboximidamide hydrochloride, to its enantioselective preparation, to a dermal formulation comprising the compound and one or more pharmaceutically acceptable excipients or carriers, and to the use of the compound as a medicament, particularly for use in the treatment of skin fibrosis.

### FIELD OF THE INVENTION

The present invention relates to (4R)-3-(4-fluoro-2-hydroxyphenyl)-4-methyl-4,5-dihydro-1H-pyrazole-1-carboximidamide hydrochloride, its enantioselective preparation, and aqueous dermal formulations thereof for topical treatment of skin fibrosis.

### BACKGROUND OF THE INVENTION

Preparation of 5-HT2B receptor antagonists as anti-fibrotic agents was described in WO2016/207231A1. Potent skin and lung anti-fibrotic effects were demonstrated in mice after oral administration, and compounds were selected for pharmaceutical development. One such selected compound was 3-(4-fluoro-2-hydroxyphenyl)-4-methyl-4,5-dihydro-1H-pyrazole-1-carboximidamide, exemplified by its hydrobromide salt (Example 50 in WO2016/207231A1).

The preparative synthetic methods described in WO2016/207231A1 could only provide racemic mixtures of chiral compounds, and chiral preparative LC methods were needed to prepare samples of the pure enantiomers. To investigate pure chiral compounds for selection and further pharmaceutical development, there is a need for effective preparative enantioselective synthetic methods to avoid ineffective and expensive chiral separation by LC methods.

Skin fibrosis, like other fibrotic conditions, is initiated by tissue injury and/or inflammation and is a natural response of wound healing. Three phases characterize wound healing, namely inflammation, proliferation, and the remodeling phase. TGF-β is a key regulator in the remodeling phase enabling the differentiation of fibroblasts to myofibroblasts, producing extracellular matrix (ECM) proteins, e.g. collagen and fibronectin, to assist with wound coverage. Prolonged inflammation, as in burn injuries, mechanical tension, and other perturbing factors, result in excessive production of ECM proteins and abnormal scarring of the skin, such as the keloid scars and hypertrophic scars.

Excessive scar formation may lead to impaired aesthetics, impaired physical function, and psychological disorders. Currently there are no standard treatments of keloids and no satisfactory treatments of hypertrophic scars (Hofmann, E. et al. Biomedicines 2023, 11, 1056). There is a very high unmet medical need for new treatment options of fibrotic disorders of the skin.

Dermal delivery of pharmaceutical compounds for the topical treatment of dermal conditions may provide certain advantages, such as high local concentration of the active compound in the skin, while systemic exposure, possibly leading to adverse effects, can be minimized. Skin penetration is, however, highly dependent on physical-chemical properties of the active compound; the main hurdle being the penetration of the stratum corneum, the keratinized lipid-rich superficial skin layer. Especially, polar compounds and ions, e.g. protonated and deprotonated bases and acids, respectively, are not able to adequately penetrate skin by passive formulation methods (Nikoli'c, I. et al. Pharmaceutics 2022, 14, 1144). Penetration can be accomplished, however, by the more elaborate and expensive active methods using physical means and special techniques, e.g. microneedles, to overcome dermal barriers.

Consequently, skin penetration of basic amine compounds is dependent on the pH of the formulation, with poor penetration at low pH. The formation of lipophilic ion-pairs, e.g. where the small chloride anion is replaced with a bulky carboxylate anion, can enhance skin penetration (Cristofoli, M. et al. Pharmaceutics 2021, 13, 909). This requires further development of the drug compound with issues related to compound stability, pharmacokinetic properties, and costs.

Since a pH 4-6 is preferred for dermal formulations, there is a challenge to formulate and deliver basic compounds, which ionize at low pH, using passive ordinary methods avoiding development of suitable ion-pairs or other methods, such as microneedle delivery.

For topical treatment of the skin, there is also a challenge to minimize the undesired transdermal permeation leading to systemic exposure with possible adverse effects.

The penetration of a compound through stratum corneum into epidermis and dermis, and permeation through the skin, can be measured in vitro using porcine or human skin. Results from these membranes are known to correlate with human in vivo data (Iliopoulos F. et al. Front. Drug. Deliv. (2022) 2:1049848).

### SUMMARY OF THE INVENTION

There are currently no truly antifibrotic medical treatment options for excessive scar formation of the skin. Topical treatment with anti-fibrotic agents can meet this huge medical need.

The anti-fibrotic 5-HT2B receptor antagonists described in WO2016/207231A1 are aminoguanidine derivatives that are basic compounds forming acid salts. The small compound aminoguanidine itself, both as base and salt, have been used in dermal formulations, however, with little data on skin penetration properties. Studies have shown that topically applied aminoguanidine hemisulfate does not penetrate to the level of skin capillaries. This implies that aminoguanidine salts do not penetrate the dermis, the target dermal tissue of the anti-fibrotic agents of the present invention.

Dermal formulations of benzylidene-aminoguanidines structurally related to Compounds 1-3, have been described (Lindahl, A. WO 2015/097513A1). To overcome the poor skin penetration properties of the ionized aminoguanidine salt derivatives, formulations of the basic forms were developed. To avoid high pH aqueous formulations, irritating to the skin, water-free solvent/oil emulsions were used.

An object of the present application is to find suitable anti-fibrotic compounds, and effective enantioselective preparative methods for such compounds, for the preparation of aqueous dermal formulations providing adequate skin penetration with little permeability through the skin. A further objective is to provide such aqueous dermal formulations for the treatment of skin fibrotic conditions.

The above objects are achieved by the subject matter described herein. In the present disclosure, the compound (R)-3-(4-fluoro-2-hydroxyphenyl)-4-methyl-4,5-dihydro-1H-pyrazole-1-carboximidamide hydrochloride (Compound 3) was identified as a stable compound with good solubility in water and other excipient solvents. The compound, formulated as an aqueous formulation and applied on skin, displayed good skin penetration properties with very little transdermal permeability. A method for preparing the compound based on steroselective hydrogenation of a key exo-methylene derivative is described. The stereochemical prefixes (R) and (4R), and (S) and (4S), respectively, are used interchangeably.

Various aspects of the invention are set out in the attached independent claims.

Further advantageous embodiments of the invention are set out in the attached dependent claims.

Applications and advantages of aspects and embodiments of the invention will be apparent from the following detailed description.

### DETAILED DESCRIPTION OF THE INVENTION

The anti-fibrotic compounds described in WO2016/207231A1 considered for development, e.g. the racemic HBr salt of Example 50 in WO2016/207231A1 (Log D −0.7), are highly polar compounds. Consequently, for dermal applications the base form (Compound 1), with expected reduced polarity, was selected for dermal formulation studies. After comparing solubilities in excipient solvents of Compound 1 with the corresponding enantiomeric base (Compound 2), isolated by chiral supercritical fluid chromatography (SFC), it was surprisingly found that Compound 2 was considerably more soluble in water, by a factor of 10-20, and consequently more suitable for aqueous formulations.

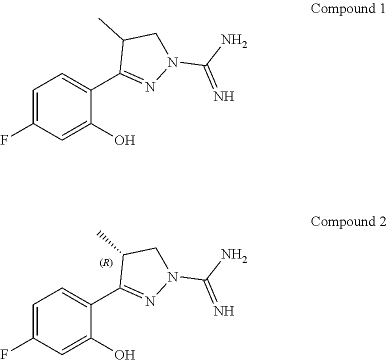

The 3D-structures and absolute stereochemistry of the two new enantiomeric compounds, (4R)- and (4S)-3-(4-fluoro-2-hydroxyphenyl)-4-methyl-4,5-dihydro-1H-pyrazole-1-carboximidamide were determined by X-ray crystallography (XRD) of the corresponding HCl salts, respectively. The R-enantiomer was identified as approx. 100 times more potent at the 5-HT2B receptor target protein and showed excellent selectivity against a number (>150) of other receptors, ion-channels, and enzymes.

Initial formulation and skin in vitro permeation tests (IVPT) using pig ear skin were performed with Compound 2 (3%, w/w) in a vehicle solvent mixture, based on solubility data, consisting of water (50%), PEG-400 (25%) and propylene glycol (25%). Compound 2 solubilities in the separate solvents were found to be 10 mg/mL in water, 63 mg/mL in PEG-400, and 35 mg/mL in propylene glycol. The solubility of Compound 2 in the vehicle solvent mixture was found to be 7.5% (w/w).

The results showed surprisingly good penetration into epidermis and dermis with only small transdermal delivery to the receiver fluid. Unfortunately, stability studies of the separate solvent solutions of Compound 2, and of the formulation, detected formation of impurities.

One known impurity of this compound class, including salt derivatives, is the urea derivative formed by hydrolysis in aqueous solutions. The rate of hydrolysis is dependent on pH with faster hydrolysis at high pH, thus acidic salt derivatives are more stable than the base forms.

The racemic HBr salt and the R-enantiomer of the HCl salt were then considered for evaluation in the IVPT test, although salt forms are expected to have poor skin penetration properties. First, to mimic long-term stability of aqueous solutions of the two acidic salts (2%, w/v), the urea derivative formation and remaining parent compound was measured by HPLC (peak areas, % of total) under forced conditions at elevated temperatures. It was surprisingly found that the racemic HBr salt (Example 50 in WO2016/207231A1) was considerably less stable with relatively high urea formation (0.6%, 2.4%, and 28.4% at 80° C., 90° C., and 100° C., respectively, after 14 days). The remaining parent HBr racemate compound in the sample stored at 100° C. was 49.7%. The enantiomeric HCl salt, on the other hand, was shown almost stable under the same conditions (0.2%, 0.7%, and 0.7% at 80° C., 90° C., and 100° C., respectively, after 14 days with >99% remaining parent compound in all samples).

The hydrolytic rate difference is surprising and not easily explained, especially since HBr is a stronger acid than HCl, although equally strong in water, but factors like the propensity to form ion-pairs or hydration layers may have some impact. Stereo-chemistry related effects, which obviously affect the solubility of the base racemic and enantiomeric forms, was ruled out, since also the stability of the racemic HCl salt was tested showing similar stability as the enantiomeric form.

The hydrolysis of guanidine to urea has been studied at 50° C. for five days and at pHs 4, 7, and 9 (Guanidinium chloride REACH registration data factsheet, EC number: 200-002-3). No hydrolysis (<10%) was detected, probably due to stable guanidinium ion at all tested pHs. The benzylidene-aminoguanidines of the present invention are less basic and hence more prone to hydrolysis at high pH. In the study, counter ion effects were assumed to be of no importance. However, guanidinium counter ion effects on hydration layers and ion-pairing have been reported (Cooper, R. J. et al. J. Phys. Chem. A 2014, 118, 30, 5657-5666) and (Hunger, J. et al. J. Phys. Chem. B 2013, 117, 2, 615-622).

Despite the enhanced polar property, and a possible urea formation, the more stable HCl salt of the enantiomeric Compound 2, (4R)-3-(4-fluoro-2-hydroxyphenyl)-4-methyl-4,5-dihydro-1H-pyrazole-1-carboximidamide hydrochloride (Compound 3), was investigated for dermal formulation development.

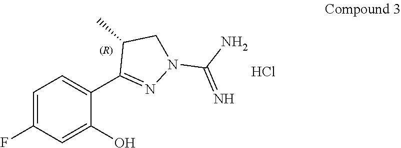

The same test procedures, and the same solvent formulation, as performed for Compound 2 were used to evaluate Compound 3. Good solubilities were demonstrated (34 mg/mL in water, 16 mg/mL in PEG-400, and 51 mg/mL in propylene glycol, and 7.1% (w/w) in the vehicle solvent mixture). The IVPT results clearly showed, like for Compound 2, surprisingly good penetration into epidermis (7.7 μg/cm²) and dermis (4.0 μg/cm²) with only small transdermal delivery to the receiver fluid (0.38 μg/cm²). In addition, Compound 3 was found stable in separate solvents during the stability studies and in the mixed solvent formulation. The Compound 3 formulation also showed a slightly acidic pH (pH 5.5), favorable for dermal formulations, while the Compound 2 formulation was found basic (pH 10.1).

Cream and gel formulations of Compound 3 were then developed. One cream formulation and one gel formulation, both with 2% of Compound 3, were tested in the IVPT assay using finite doses without occlusion.

Both formulations, cream and gel, delivered Compound 3 with excellent penetration into epidermis (20.7 and 11.5 μg/cm², respectively) and dermis (8.1 and 3.1 μg/cm², respectively) with high dose fractions; >8% and >4%, respectively. The transdermal delivery to the receiver fluid was very low for both formulations: 0.20 μg/cm² from the cream; and 0.03 μg/cm² from the gel.

Next, the long-term stabilities, up to nine months reported, of the cream and gel formulations stored at 25° C., 30° C., and 40° C. were examined. Results showed that he initial Compound 3 concentration (2.03%) remained stable in all samples (1.97-2.14%). Sum of related impurities, initially 0.11% in both formulations, were also stable in all cream samples except for samples stored at 40° C., which increased slightly over time to 0.53% at 9 months. A slight increase of related impurities was also seen in the gel samples stored at 30° C. and 40° C., showing 0.17% and 0.53% at 9 months, respectively.

The passive dermal formulations of Compound 3, a salt of a polar basic compound with expected low skin penetration ability, described in the present invention, have been shown to effectively deliver the anti-fibrotic Compound 3 to the target skin tissue without notable skin permeation, minimizing systemic exposure. Compound 3 and the formulations thereof, have also been shown to be very stable even at elevated temperatures and over long-term storage. This implies that the formulations are well suited for dermal topical delivery of Compound 3.

The preparation of racemic compounds described in WO2016/207231A1 included 4-nor- and 4-Me-2-pyrazolines, using acetophenones and propiophenones, respectively, as starting materials in the Mannich reaction (Scheme 1).

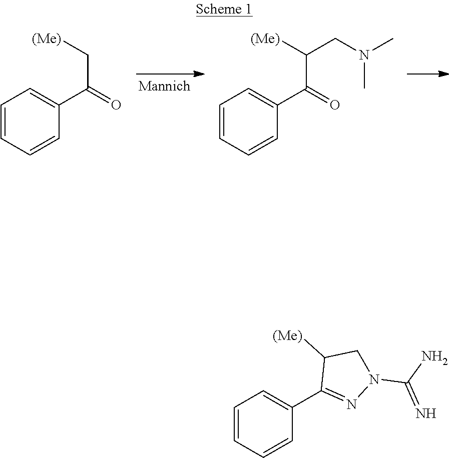

It was found that small amounts of bis-adducts were formed as byproducts in the Mannich reaction with acetophenones, and that the bis-adduct can further react by elimination of dialkylamine to provide a 3-dialkylylamino-2-methylene-propiophenone (Scheme 2).

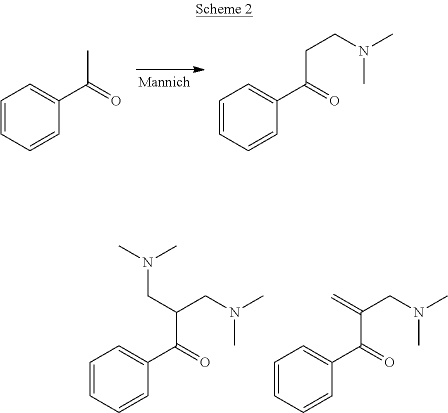

This product can potentially be reacted with aminoguanidine to give a 4-methylene-2-pyrazoline, a suitable substrate for enantioselective hydrogenation, providing chiral products (Scheme 3). The regio-chemistry of the ring-closure is especially demanding; the 3-dialkylylamino-2-methylene-propiophenone starting material contains three electrophilic carbons (carbonyl and two beta-carbons) and the aminoguanidine contains four nitrogen atoms, the two hydrazine nitrogens being the most nucleophilic.

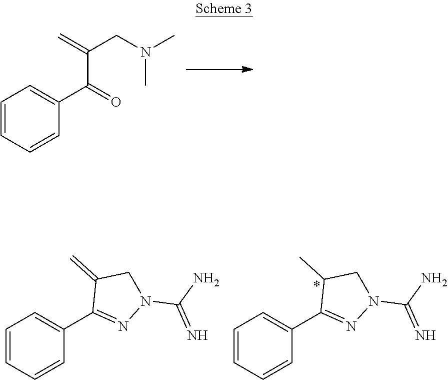

The synthetic plan outlined above was developed into an effective preparative method for the synthesis of (R)- and (S)-3-(4-fluoro-2-hydroxyphenyl)-4-methyl-4,5-dihydro-1H-pyrazole-1-carboximidamide, respectively (Scheme 4, (R)-isomers shown). It is understood that similar chiral 3-phenyl-4-methyl-4,5-dihydro-1H-pyrazole-1-carboximidamide derivatives can be prepared by basically the same methods.

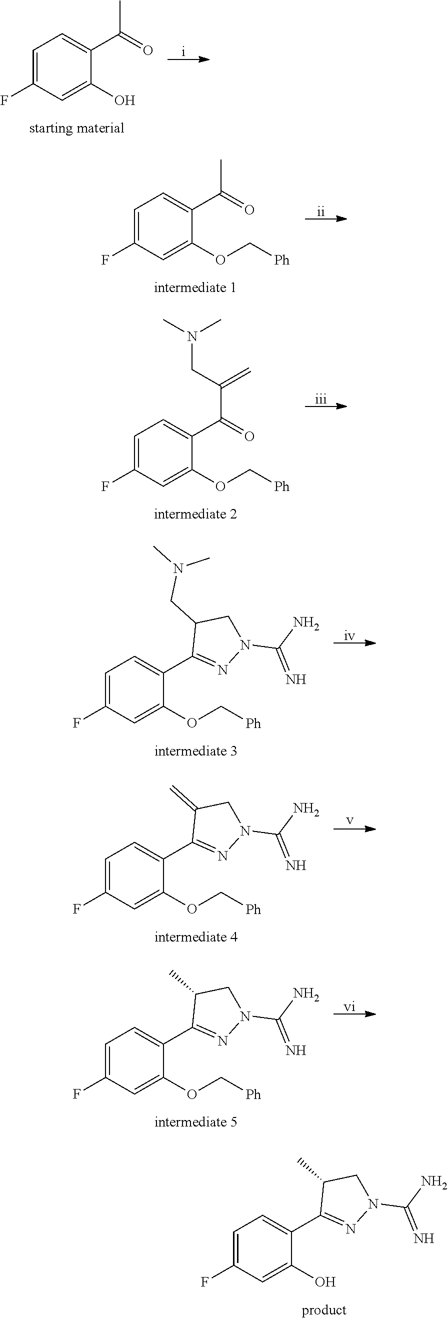

Benzylation (i)—The phenolic moiety of the starting material, 4′-fluoro-2′-hydroxyacetophenone, is protected by benzylation to avoid intramolecular ring-closure with the enone moiety of intermediate 2. Benzylation can be achieved by reacting the phenolic starting material with benzyl bromide in an appropriate solvent like MeCN or DMF in the presence of a base, e.g. K₂CO₃. Alternative protecting groups, stable through the synthetic route ii-v, may be used, e.g. other ethers and silyl ethers.

Mannich reaction (ii)—This reaction, a “double Mannich reaction”, provides two —CH₂NMe₂ moieties, one of which can eliminate HNMe₂, to give the enone structure. The starting acetophenone can be reacted with ready-made Mannich reagents, e.g. the Eschenmoser's salt (H₂C═NMe₂⁺ I⁻), or alternatively, with in situ generated reagents prepared under acidic conditions from a formaldehyde equivalent, e.g. paraformaldehyde or 1,3-dioxolane, and a dialkylamine or a dialkylammonium salt, e.g. pyrrolidine or H₂NMe₂Cl, respectively.

Ring-closure with aminoguanidine (iii)—Most conveniently, aminoguanidine hydrochloride is used in the ring-closure, which can be performed both under acidic, e.g. in refluxing EtOH, and basic conditions, and in aqueous or non-aqueous solvents, typically at temperatures ranging from room temperature to 80° C. Under basic conditions, aq. NaOH can be used as base in solvents like THF, MeCN, EtOH and water. The intermediate 3 is most conveniently isolated as a salt, e.g. as a bis-trifluoroacetic acid (bis-TFA) salt or bis-methansulfonic acid salt, which can be used as starting material in the following Cope-elimination.

Cope-elimination (iv)—This reaction is an oxidation-elimination process, wherein the trialkylamine moiety is oxidized to give an N-oxide, which eliminates N-hydroxy-dialkylamine to provide the exo-methylene moiety. Per-acids, e.g. MCPBA, or inorganic oxides, e.g. potassium peroxymonosulfate, are often used as oxidation agents, optionally generated in situ by oxidation with e.g. H₂O₂ or O₂ (g). (Xingwei Cai et al., Asian Journal of Chemistry; Vol. 24, No. 9 (2012), 3781-3784). The N-oxide of intermediate 3 can be isolated before elimination, or the two-step process can be performed as a one-pot procedure. The elimination process is affected by heating the N-oxide in an appropriate solvent, e.g. EtOAc, MeOH or CH₂Cl₂. Temperatures vary with the substrates and solvents. For the N-oxide of intermediate 3, elimination occurs conveniently at 40-80° C.

Another option for the elimination processes is via N-alkylation to give a tetra-alkylammonium salt that can eliminate trialkyl-amine under basic conditions.

Enantioselective hydrogenation (v)—Transition metal catalysis for asymmetric hydrogenation of double bonds, e.g. a C═C double bond, using hydrogen gas may be a powerful way to generate chiral products (Biosca, M. et al. Asymmetric hydrogenation in industry, Advances in Catalysis, Volume 68, 2021, Pages 341-383). Rh and Ru in complex with chiral diphosphine ligands are among the most common catalysts and many ligands are commercially available in both enantiomeric forms, providing access to both R- and S-enantiomeric products. Normally, many different reaction conditions, including the catalyst system, are tested to identify adequate and scalable preparative methods that can provide an acceptable enantiomeric purity of the product. In addition to the selection of metal source and ligand, catalyst loading, concentration, solvent(s), additives, temperature, and hydrogen pressure should also be considered. For basic compounds, salt formation by addition of acids may have a large impact on the results. Alternatively, the preformed salt of the staring material can be used in the reaction.

Asymmetric hydrogenation of intermediate 4 can be performed using a catalyst prepared from a Rh source, e.g. [Rh(nbd)₂]/BF₄ (bis(norbornadiene)rhodium(I) tetrafluoroborate), and a diphosphine ligand, e.g. SL-A242, SL-WO09, SL-J004, SL-J006, SL-J007, SL-J502, SL-J505, SL-J212, SL-C008, SL-M002, or SL-T002, ligands which all are commercially available in both enantiomeric forms. The intermediate 4 is preferably used as a salt form, formed from e.g. the acids AcOH, CF₃CO₂H (TFA), MeSO₂OH, H₂SO₄, KHSO₄, or H₃PO₄ or alternatively in combination with an acid as an additive, in an appropriate solvent, e.g. MeOH, EtOH, I—PrOH, THF, 2-Me-THF or dioxane.

Debenzylation (vi)—Catalytic hydrogenation in inert solvents, e.g. alcohols, THF or toluene, with Pd/C as catalyst and H₂ (g) as hydrogen source is a standard procedure. Alternative hydrogen sources are e.g. ammonium formate and c-hexadiene. After the reaction is complete, the product remains in solution and is separated from the Pd/C by filtration. Since both intermediate 5 and the product are polar compounds, the hydrogenation should be performed in water or alcohols, such as methanol.

Importantly, it was surprisingly found that the base form of the enantiomeric product is considerably more soluble in water (20 times) and methanol than the racemic product. Thus, the debenzylation can be performed using the base form of the chiral intermediate 5 to provide the soluble enantiomeric debenzylated product (Compound 2), which then directly can undergo chiral enrichment using the chiral acid D-DpTTA, as described below, by trituration in relatively small volumes (10-20 vol eq.). This is contrary to a method where the racemate is used, requiring HCl addition to keep the racemic debenzylated product in solution, followed by filtration to remove the Pd/C catalyst, and re-basification to isolate the racemic base. The racemic base can then enter a resolution process using the chiral acid D-DpTTA. However, very large volumes are required (150-200 vol. eq.) in the resolution of the racemic product. There is a great advantage of using the enantiomeric product both as a soluble product in the debenzylation reaction, but more importantly in the chiral enrichment/resolution process.

Debenzylation of intermediate 5 can also be performed under acidic solvolytic conditions, e.g. in aq. conc. HCl mixtures with MeOH, EtOH or AcOH or in HBr/AcOH.

The enantiomeric purity of the target product can, if desired, be further improved by recrystallization of intermediate 5 and/or the product, preferably as salt derivatives, e.g. HCl salts, or as diastereomeric salts, e.g the di-O,O′-p-toloyl-D-tartaric acid (D-DpTTA) salt of the (R)-isomeric product. Both the D- and the L-isomer of the di-O,O′-p-toloyl-tartaric acid, (2S, 3S) and (2R, 3R), respectively, are commercially available. The L-isomer can thus be used to improve the enantiomeric purity of the (S)-isomer of the product.

According to a first aspect of the invention, the above mentioned and other objects are achieved by the compound (4R)-3-(4-fluoro-2-hydroxyphenyl)-4-methyl-4,5-dihydro-1H-pyrazole-1-carboximidamide hydrochloride (the compound of Formula 3).

The inventive compound is preferably provided in an enantiomerically pure or enantiomerically enriched form. In some embodiments, the compound has an enantiomeric purity of at least about 80%, such as at least about 90%, such as at least about 95%, or such as at least about 97%. In some embodiments, the inventive compound is provided in a composition or a formulation comprising the compound in an enantiomerically pure or enantiomerically enriched form.

As used herein, the term “enantiomeric purity” refers to the prevalence of one enantiomer of a compound over the opposite enantiomer of the compound. A typical enantiomerically pure compound, composition or formulation, comprises greater than about 80% by weight of one enantiomer of the compound and less than about 20% by weight of the opposite enantiomer of the compound, more preferably greater than about 90% by weight of one enantiomer of the compound and less than about 10% by weight of the opposite enantiomer of the compound, even more preferably greater than about 95% by weight of one enantiomer of the compound and less than about 5% by weight of the opposite enantiomer of the compound, and most preferably greater than about 97% by weight of one enantiomer of the compound and less than about 3% by weight of the opposite enantiomer of the compound.

The inventive compound has been found to be useful in dermal formulations and particularly in aqueous dermal formulations.

According to a second aspect of the invention, there is provided a dermal formulation comprising the compound (4R)-3-(4-fluoro-2-hydroxyphenyl)-4-methyl-4,5-dihydro-1H-pyrazole-1-carboximidamide hydrochloride (the compound of Formula 3) and one or more pharmaceutically acceptable excipients or carriers.

The inventive dermal formulation preferably comprises (4R)-3-(4-fluoro-2-hydroxyphenyl)-4-methyl-4,5-dihydro-1H-pyrazole-1-carboximidamide hydrochloride in an enantiomerically pure or enantiomerically enriched form.

In some embodiments of the second aspect at least about 80%, such as at least about 90%, such as at least about 95%, or such as at least about 97% of the total content of 3-(4-fluoro-2-hydroxyphenyl)-4-methyl-4,5-dihydro-1H-pyrazole-1-carboximidamide hydrochloride in the formulation consists of (4R)-3-(4-fluoro-2-hydroxyphenyl)-4-methyl-4,5-dihydro-1H-pyrazole-1-carboximidamide hydrochloride.

As used herein, the term “dermal formulation” refers to a composition or mixture specifically designed and formulated for topical application on the skin. It is intended to deliver active ingredients, provide therapeutic effects, enhance skin health, or improve cosmetic appearance through direct contact with the skin.

The term “dermal formulation” encompasses various types of formulations, including creams, lotions, gels, ointments, sprays, foams, patches, or any other form suitable for application to the skin. These formulations typically consist of a combination of active ingredients, excipients, vehicles, penetration enhancers, emulsifiers, stabilizers, and other components that contribute to the desired properties, stability, and efficacy when applied topically.

In some embodiments, the dermal formulation is an aqueous formulation. As used herein, the term “aqueous formulation” refers to a formulation, composition or mixture that contains water as the primary solvent or dispersion medium.

In some embodiments, the dermal formulation comprises about ≥20% (w/w) of water, such as about ≥30% (w/w) of water, or such as about ≥40% (w/w) of water.

In some embodiments, the dermal formulation comprises about 50-98% (w/w) of water, such as about 50-95% (w/w) of water, or such as about 50-90% (w/w) of water.

In some embodiments, the dermal formulation further comprises about 1-30% (w/w) propylene glycol, such as about 5-25% (w/w) propylene glycol.

In some embodiments, the dermal formulation further comprises about 1-20% (w/w) glycerol, such as about 5-15% (w/w) glycerol.

In some embodiments, the dermal formulation comprises or consists of a solution or suspension of the inventive compound in a liquid, preferably aqueous, carrier. The solution or suspension may be designed for topical application on the skin or mucous membranes. The solution or suspension may be specifically formulated to provide therapeutic effects when applied to the affected area.

In some embodiments, the dermal formulation is a cream. As used herein, the term “cream” refers to a semisolid formulation designed for topical application on the skin or mucous membranes, containing the inventive compound suspended or dissolved in a suitable base or vehicle. The cream is specifically formulated to provide therapeutic effects when applied to the affected area. Methods and components for formulating dermal cream formulations are known and readily available to the skilled person.

In dermal cream formulations for delivering pharmaceutical agents, the dermal formulation typically comprises two phases: the aqueous phase and the lipid (oil) phase. Each phase contains specific types of ingredients that contribute to the overall properties and functionality of the cream. In dermal creams formulated to deliver pharmaceutical agents to patients, the aqueous and lipid phases contain specific components tailored to enhance the stability, efficacy, and delivery of the active ingredients.

Common components for the aqueous phase include, but are not limited to:

Water: The primary solvent, used as a base to dissolve water-soluble ingredients.

Active Ingredients: Hydrophilic active pharmaceutical ingredients (APIs) that dissolve in water.

Solubilizers: Such as polyethylene glycol (PEG) and polysorbates (e.g. polysorbate 60), which help dissolve and stabilize poorly water-soluble drugs in the aqueous phase.

Humectants: Such as glycerol, propylene glycol, and sorbitol, which attract and retain moisture, enhancing skin hydration and helping in the penetration of active ingredients.

Thickeners and Gelling Agents: Such as carbomers, xanthan gum, and hydroxyethylcellulose, which provide viscosity and texture to the aqueous phase.

Chelating Agents: Such as EDTA (ethylenediaminetetraacetic acid), which bind metal ions that could destabilize the formulation.

Buffers: Such as citric acid, sodium citrate, and phosphates, which help maintain the pH of the cream within an optimal range.

Preservatives: Such as parabens (methylparaben, propylparaben), benzyl alcohol, benzalkonium chloride, and phenoxyethanol, which prevent microbial growth and prolong shelf life.

Common components for the lipid phase include, but are not limited to:

Oils: Such as mineral oil, plant oils (e.g., jojoba oil, almond oil), and synthetic oils (e.g., isopropyl myristate), which help to form an occlusive layer on the skin.

Waxes: Such as beeswax, carnauba wax, and paraffin, which provide structure and stability to the cream.

Fatty Alcohols: Such as cetyl alcohol, stearyl alcohol, and cetearyl alcohol, which act as thickeners and stabilizers.

Emollients: Such as lanolin, petrolatum, and squalane, which soften and smooth the skin, enhancing the delivery of the active pharmaceutical ingredient.

Emulsifiers: Such as glyceryl stearate, sorbitane monostearate, PEG-100 stearate, and polysorbates, which stabilize the mixture of oil and water phases, ensuring a consistent and homogenous product.

Silicones: Such as dimethicone and cyclopentasiloxane, which enhance spreadability and provide a smooth, silky texture.

Active Ingredients: Lipophilic active pharmaceutical ingredients that dissolve in oils.

Penetration Enhancers: Such as oleic acid and isopropyl myristate, which help improve the absorption of the active ingredients through the skin.

The components of the dermal cream formulation are selected to optimize the delivery of the pharmaceutical agent, ensuring the cream is effective, stable, and pleasant to use. The combination of aqueous and lipid phases allows for the incorporation of a wide range of active ingredients, tailored to the specific therapeutic needs of the patient.

In some embodiments, the aqueous phase comprises water, the active compound (e.g., Compound 3), at least one solubilizer (e.g., a polysorbate, such as polysorbate 60), at least one humectant (e.g., glycerol or propylene glycol), at least one emollient (2-Octyl-1-dodecanol), and at least one emulsifier (e.g., sorbitane monostearate). Some of the components may have more than one function.

In some embodiments, the lipid phase comprises at least one wax (e.g. cetyl palmitate), at least one fatty alcohol (e.g., cetyl alcohol, stearyl alcohol, or a combination thereof (cetostearyl alcohol)), at least one emollient (2-Octyl-1-dodecanol), and at least one emulsifier (e.g., sorbitane monostearate). Some of the components may have more than one function.

In some embodiments, the dermal formulation is a gel. As used herein, the term “gel”, as described in patent language, refers to a semisolid formulation designed for topical or mucosal application, characterized by its gel-like consistency and ability to retain shape under minimal shear stress. The gel composition comprises the inventive compound and a gelling agent or matrix, which imparts the desired gel-like properties to the formulation. Methods and components for formulating dermal gel formulations are known and readily available to the skilled person.

In dermal gel formulations for delivering pharmaceutical agents, the formulation typically comprises at least water, the active compound, and a gelling agent.

Common components for gel formulations include, but are not limited to: Water: The primary solvent, used as a base to dissolve water-soluble ingredients and providing the base for the gel.

Active Ingredients: Hydrophilic active pharmaceutical ingredients (APIs) that dissolve in water.

Gelling Agents: For example carbomers (e.g., Carbopol), cellulose derivatives (e.g., hydroxyethyl cellulose or hydroxypropyl methylcellulose), and poloxamers, which provide viscosity and gel structure.

Solubilizers and Surfactants: Such as polysorbates (e.g., polysorbate 20 or polysorbate 80), which help dissolve hydrophobic active ingredients in the aqueous base.

Humectants: Such as glycerol, and propylene glycol, which attract and retain moisture, enhancing skin hydration and helping in the penetration of active ingredients.

Penetration Enhancers: Such as oleic acid and isopropyl myristate, which help improve the absorption of the active ingredients through the skin.

Emollients: Such as propylene glycol and isopropyl myristate.

Buffers: Such as citric acid, sodium citrate, and phosphates, which help maintain the pH of the gel within an optimal range.

Chelating agents: Such as EDTA (Ethylenediaminetetraacetic acid), which stabilizes the formulation by binding metal ions.

Preservatives: Such as parabens (methylparaben, propylparaben), benzyl alcohol, benzalkonium chloride, and phenoxyethanol, which prevent microbial growth and prolong shelf life.

The components of the dermal gel formulation are chosen to ensure the gel is effective, stable, and comfortable to apply, providing the intended therapeutic effect while being pleasant for the patient to use.

In some embodiments, the dermal gel formulation comprises water, the active compound (e.g., Compound 3), and at least one gelling agent (e.g., hydroxyethylcellulose). In some embodiments, the dermal gel formulation comprises water, the active compound (e.g., Compound 3), at least one gelling agent (e.g., a carbomer, such as carbopol), at least one humectant (e.g., glycerol or propylene glycol), at least one chelating agent (e.g., EDTA (Ethylenediaminetetraacetic acid). Some of the components may have more than one function.

According to a third aspect of the invention, there is provided the compound (4R)-3-(4-fluoro-2-hydroxyphenyl)-4-methyl-4,5-dihydro-1H-pyrazole-1-carboximidamide hydrochloride (the compound of Formula 3) for use as a medicament. In some embodiments, the compound is provided in a composition or a formulation comprising the compound in an enantiomerically pure or enantiomerically enriched form.

According to a fourth aspect of the invention, there is provided the compound (4R)-3-(4-fluoro-2-hydroxyphenyl)-4-methyl-4,5-dihydro-1H-pyrazole-1-carboximidamide hydrochloride (the compound of Formula 3) for use in the treatment of skin fibrosis. In some embodiments, the compound is provided in a composition or a formulation comprising the compound in an enantiomerically pure or enantiomerically enriched form.

In some embodiments, the skin fibrosis is caused by a disease or skin inflammation, a natural ageing process, scarring caused by burn injuries, by surgery, by plastic surgery, or by other skin injuries, acne, or keloids.

In some embodiments, the skin fibrosis is caused by the disease systemic sclerosis.

In some embodiments, the skin fibrosis is caused by burn injuries.

In some embodiments, the skin fibrosis is caused by surgery or plastic surgery. In some embodiments, the skin fibrosis is caused by acne.

In some embodiments, the skin fibrosis is caused by keloids.

According to another aspect of the invention, there is provided the use of the compound (4R)-3-(4-fluoro-2-hydroxyphenyl)-4-methyl-4,5-dihydro-1H-pyrazole-1-carboximidamide hydrochloride (the compound of Formula 3) in the manufacture of a medicament useful in treatment of skin fibrosis. In some embodiments, the compound is provided in a composition or a formulation comprising the compound in an enantiomerically pure or enantiomerically enriched form.

According to another aspect of the invention, there is provided a method of treating skin fibrosis comprising administering a therapeutically effective amount of the compound (4R)-3-(4-fluoro-2-hydroxyphenyl)-4-methyl-4,5-dihydro-1H-pyrazole-1-carboximidamide hydrochloride (the compound of Formula 3) to a patient in need thereof. In some embodiments, the compound is provided in a composition or a formulation comprising the compound in an enantiomerically pure or enantiomerically enriched form.

According to another aspect of the invention, there is provided a method for preparing (R)- or (S)-3-(4-fluoro-2-hydroxyphenyl)-4-methyl-4,5-dihydro-1H-pyrazole-1-carboximidamide or a pharmaceutically acceptable salt thereof; the method comprising:
a) providing the compound 1-(2-(benzyloxy)-4-fluorophenyl)-2-((dimethylamino)methyl)prop-2-en-1-one;

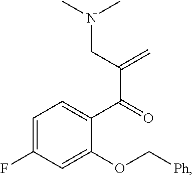 [SMILES: C=C(CN(C)C)C(=O)c1ccc(F)cc1OCc1ccccc1]

b) subjecting the compound 1-(2-(benzyloxy)-4-fluorophenyl)-2-((dimethylamino)methyl)prop-2-en-1-one to a ring closure process with aminoguanidine or aminoguanidine hydrochloride to obtain the compound 3-(2-(benzyloxy)-4-fluorophenyl)-4-((dimethylamino)methyl)-4,5-dihydro-1H-pyrazole-1-carboximidamide, optionally as a bis-trifluoroacetic acid salt,

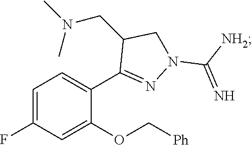 [SMILES: CN(C)CC1CN(C(=N)N)N=C1c1ccc(F)cc1OCc1ccccc1]

c) subjecting the compound 3-(2-(benzyloxy)-4-fluorophenyl)-4-((dimethylamino)methyl)-4,5-dihydro-1H-pyrazole-1-carboximidamide bis-trifluoroacetic acid salt to an elimination process to obtain the compound 3-(2-(benzyloxy)-4-fluorophenyl)-4-methylene-4,5-dihydro-1H-pyrazole-1-carboximidamide, optionally as a salt with an acid selected from CF₃CO₂H and MeS(O)₂OH

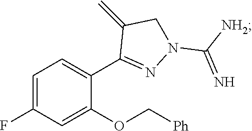 [SMILES: C=C1CN(C(=N)N)N=C1c1ccc(F)cc1OCc1ccccc1]

d) subjecting the compound 3-(2-(benzyloxy)-4-fluorophenyl)-4-methylene-4,5-dihydro-1H-pyrazole-1-carboximidamide, optionally as a salt with an acid selected from CF₃CO₂H and MeS(O)₂OH, to enantioselective hydrogenation to obtain the compound (R)-3-(2-(benzyloxy)-4-fluorophenyl)-4-methyl-4,5-dihydro-1H-pyrazole-1-carboximidamide, or (S)-3-(2-(benzyloxy)-4-fluorophenyl)-4-methyl-4,5-dihydro-1H-pyrazole-1-carboximidamide, optionally as a salt with an acid selected from CF₃CO₂H and MeS(O)₂OH

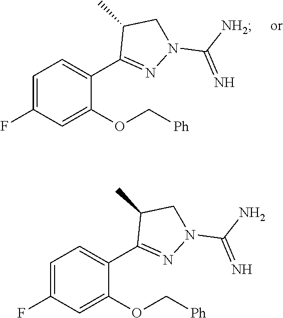 [SMILES: C[C@@H]1CN(C(=N)N)N=C1c1ccc(F)cc1OCc1ccccc1 ; C[C@H]1CN(C(=N)N)N=C1c1ccc(F)cc1OCc1ccccc1]

e) subjecting the compound (R)-3-(2-(benzyloxy)-4-fluorophenyl)-4-methyl-4,5-dihydro-1H-pyrazole-1-carboximidamide, or (S)-3-(2-(benzyloxy)-4-fluorophenyl)-4-methyl-4,5-dihydro-1H-pyrazole-1-carboximidamide, to deprotection, and optionally forming a pharmaceutically acceptable salt of the deprotected compound to obtain the compound (R)-3-(4-fluoro-2-hydroxyphenyl)-4-methyl-4,5-dihydro-1H-pyrazole-1-carboximidamide, or (S)-3-(2-(benzyloxy)-4-fluorophenyl)-4-methyl-4,5-dihydro-1H-pyrazole-1-carboximidamide, or a pharmaceutically acceptable salt thereof.

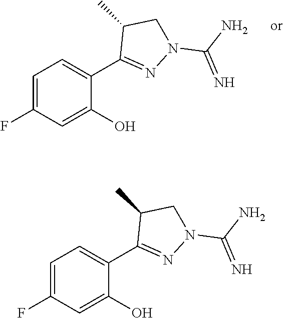 [SMILES: C[C@@H]1CN(C(=N)N)N=C1c1ccc(F)cc1O ; C[C@H]1CN(C(=N)N)N=C1c1ccc(F)cc1O]

As used herein, the term “pharmaceutically acceptable salt” refers to a chemical compound formed by the reaction between a pharmaceutically active compound and an acid or base, which is suitable for use in pharmaceutical formulations and is generally recognized as safe for human or veterinary administration. The term “pharmaceutically acceptable” indicates that the salt is compatible with the intended pharmaceutical application, including but not limited to solubility, stability, bioavailability, and lack of toxicity or adverse effects. Furthermore, the salt should meet the regulatory requirements set forth by relevant authorities, such as the U.S. Food and Drug Administration (FDA) or the European Medicines Agency (EMA), ensuring its suitability for use in the development, manufacturing, and commercialization of pharmaceutical products. Such salts include acid addition salts, which can be obtained by reaction of the free base of the pharmaceutically active compound with inorganic acids such as hydrochloric acid, hydrobromic acid, nitric acid, phosphoric acid, sulfuric acid, and perchloric acid and the like, or with organic acids such as acetic acid, oxalic acid, (D) or (L) malic acid, maleic acid, methanesulfonic acid, ethanesulfonic acid, p-toluenesulfonic acid, salicylic acid, tartaric acid, citric acid, succinic acid or malonic acid and the like.

In a preferred embodiment the prepared compound is:
(R)-3-(4-fluoro-2-hydroxyphenyl)-4-methyl-4,5-dihydro-1H-pyrazole-1-carboximidamide hydrochloride; or
(S)-3-(4-fluoro-2-hydroxyphenyl)-4-methyl-4,5-dihydro-1H-pyrazole-1-carboximidamide hydrochloride.

As used herein, the term “enantioselective hydrogenation” refers to a chemical process for selectively reducing a prochiral or chiral compound using hydrogen gas (H₂), or alternative hydrogen donor reagents, in the presence of a catalyst to produce a chiral product with a specific desired stereoisomeric configuration. The term “enantioselective” signifies the ability of the hydrogenation process to favor the formation of one enantiomer over the other, leading to the production of a single enantiomer or an enriched mixture of a specific enantiomer. The enantioselective hydrogenation process typically involves the use of a chiral catalyst, which imparts chirality to the reaction, thereby influencing the stereochemical outcome. The catalyst can be a transition metal complex, such as those based on rhodium, ruthenium, or iridium, among others, or other types of catalysts, such as enzymes or organocatalysts. The chiral catalyst selectively interacts with the substrate, facilitating the hydrogenation reaction in a manner that favors the formation of a specific enantiomer, while suppressing or inhibiting the formation of its enantiomeric counterpart.

The starting compound, 1-(2-(benzyloxy)-4-fluorophenyl)-2-((dimethylamino)methyl)prop-2-en-1-one, provided in step a) of the inventive method can be prepared by known methods described in the literature. It is understood that protecting groups (PG) may be employed and that addition, deletion, or transformation of substituents may be part of the synthetic routes.

The enantiomeric purity of the compound (R)- or (S)-3-(4-fluoro-2-hydroxyphenyl)-4-methyl-4,5-dihydro-1H-pyrazole-1-carboximidamide or a pharmaceutically acceptable salt thereof can, if desired, be further improved by recrystallization of the compounds, preferably as salt derivatives, e.g. HCl salts, or as diastereomeric salts.

In some embodiments, the inventive method further comprises:
f) subjecting the compound (R)- or (S)-3-(4-fluoro-2-hydroxyphenyl)-4-methyl-4,5-dihydro-1H-pyrazole-1-carboximidamide or a pharmaceutically acceptable salt thereof obtained in e) to diastereomeric salt formation with di-O,O′-p-toloyl-D-tartaric acid or di-O,O′-p-toloyl-L-tartaric acid, respectively, followed by salt exchange to obtain the compound (R)- or (S)-3-(4-fluoro-2-hydroxyphenyl)-4-methyl-4,5-dihydro-1H-pyrazole-1-carboximidamide with increased enantiomeric purity or a pharmaceutically acceptable salt thereof.

As used herein the term “diastereomeric salt formation” refers to the formation of salts from a chiral compound and an acid or base, resulting in the generation of diastereomers. Diastereomers are stereoisomers that are not mirror images of one another, but possess different configurations at one or more chiral centers, while maintaining the same connectivity of atoms. The term “diastereomeric salt” indicates that the salt is composed of diastereomers rather than enantiomers.

As used herein the term “salt exchange” refers to a chemical process involving the transformation of a salt from one counterion to another. It involves the exchange of ions within a salt while retaining the overall molecular structure of the compound.

In some embodiments of the inventive method, the obtained compound (R)- or (S)-3-(4-fluoro-2-hydroxyphenyl)-4-methyl-4,5-dihydro-1H-pyrazole-1-carboximidamide or a pharmaceutically acceptable salt thereof has an enantiomeric purity of at least about 80%, such as at least about 90%, such as at least about 95%, or such as at least about 97%.

As used herein, the term “enantiomeric purity” refers to the prevalence of one enantiomer of a compound over the opposite enantiomer of the compound. A typical optically pure compound comprises greater than about 80% by weight of one enantiomer of the compound and less than about 20% by weight of the opposite enantiomer of the compound, more preferably greater than about 90% by weight of one enantiomer of the compound and less than about 10% by weight of the opposite enantiomer of the compound, even more preferably greater than about 95% by weight of one enantiomer of the compound and less than about 5% by weight of the opposite enantiomer of the compound, and most preferably greater than about 97% by weight of one enantiomer of the compound and less than about 3% by weight of the opposite enantiomer of the compound.

The inventive method described herein further provides a number of intermediates useful for obtaining the desired compounds (R)- or (S)-3-(4-fluoro-2-hydroxyphenyl)-4-methyl-4,5-dihydro-1H-pyrazole-1-carboximidamide or a pharmaceutically acceptable salt thereof. Each of these intermediates are relevant as starting materials or intermediates for preparing the desired compounds (R)- or (S)-3-(4-fluoro-2-hydroxyphenyl)-4-methyl-4,5-dihydro-1H-pyrazole-1-carboximidamide or a pharmaceutically acceptable salt thereof.

According to another aspect of the invention, there is provided a compound selected from:
1-(2-(Benzyloxy)-4-fluorophenyl)-2-((dimethylamino)methyl)prop-2-en-1-one;
3-(2-(Benzyloxy)-4-fluorophenyl)-4-((dimethylamino)methyl)-4,5-dihydro-1H-pyrazole-1-carboximidamide;
3-(2-(Benzyloxy)-4-fluorophenyl)-4-methylene-4,5-dihydro-1H-pyrazole-1-carboximidamide;
(R)-3-(2-(Benzyloxy)-4-fluorophenyl)-4-methyl-4,5-dihydro-1H-pyrazole-1-carboximidamide;
(S)-3-(2-(Benzyloxy)-4-fluorophenyl)-4-methyl-4,5-dihydro-1H-pyrazole-1-carboximidamide;
and salts thereof formed with acids selected from HCl, AcOH, H₂SO₄, H₃PO₄, CF₃CO₂H, and MeS(O)₂OH.

In some embodiments, the inventive method comprises subjecting the compound (R)- or (S)-3-(4-fluoro-2-hydroxyphenyl)-4-methyl-4,5-dihydro-1H-pyrazole-1-carboximidamide or a pharmaceutically acceptable salt thereof to diastereomeric salt formation with di-0,O′-p-toloyl-D-tartaric acid or di-O,O′-p-toloyl-L-tartaric acid, respectively. Thus, according to another aspect, there is provided a compound selected from the group consisting of:
(R)-3-(4-fluoro-2-hydroxyphenyl)-4-methyl-4,5-dihydro-1H-pyrazole-1-carboximidamide di-O,O′-p-toloyl-D-tartaric acid salt; and
(S)-3-(4-fluoro-2-hydroxyphenyl)-4-methyl-4,5-dihydro-1H-pyrazole-1-carboximidamide di-O,O′-p-toloyl-L-tartaric acid salt.

### Itemized List of Embodiments

E1. The compound (R)-3-(4-fluoro-2-hydroxyphenyl)-4-methyl-4,5-dihydro-1H-pyrazole-1-carboximidamide or a pharmaceutically acceptable salt thereof.

E2. The compound of E1, wherein the compound is 3-(4-fluoro-2-hydroxyphenyl)-4-methyl-4,5-dihydro-1H-pyrazole-1-carboximidamide hydrochloride.

E3. A compound according to E1 or E2, wherein the compound has an enantiomeric purity of at least about 80%, such as at least about 90%, such as at least about 95% or such as at least about 97%.

E4. A dermal formulation comprising the compound according to any one of E2-E3 and one or more pharmaceutically acceptable excipients or carriers.

E5. The dermal formulation according to E4, wherein the dermal formulation is an aqueous formulation.

E6. The dermal formulation according to any one of E4-E5, wherein the dermal formulation comprises about 20% (w/w) of water, such as about 30% (w/w) of water, or such as about 40% (w/w) of water.

E7. The dermal formulation according to any one of E4-E6, wherein the dermal formulation comprises about 50-98% (w/w) of water, such as about 50-95% (w/w) of water, or such as about 50-90% (w/w) of water.

E8. The dermal formulation according to any one of E4-E7, wherein the dermal formulation further comprises about 1-30% (w/w) propylene glycol, such as about 5-25% (w/w) propylene glycol.

E9. The dermal formulation according to any one of E4-E8, wherein the dermal formulation further comprises about 1-20% (w/w) glycerol, such as about 5-15% (w/w) glycerol.

E10. The dermal formulation according to any one of E4-E7, wherein the dermal formulation comprises about 2% (w/w) compound of E2, about 53.65% (w/w) water, about 0.97% (w/w) polysorbate 60, about 15% (w/w) propylene glycol, about 10% (w/w) glycerol, about 1.29% (w/w) sorbitane monostearate, about 1.93% (w/w) cetyl palmitate, about 6.45% (w/w) cetostearyl alcohol, and about 8.71% (w/w) 2-octyl-1 dodecanol.

E11. The dermal formulation according to any one of E4-E7, wherein the dermal formulation comprises about 2% (w/w) compound of E2, about 15% (w/w) propylene glycol, about 0.05% (w/w) EDTA, about 0.005% (w/w) benzalkonium chloride, about 81.745% (w/w) water, about 0.90% (w/w) Carbopol 974 P, and about 0.30% (w/w) sodium hydroxide.

E12. The dermal formulation according to any one of E4-E7, wherein the dermal formulation comprises about 2% (w/w) compound of E2, about 96% (w/w) water, and about 2% (w/w) hydroxyethylcellulose.

E13. The dermal formulation according to any one of E4-E7, wherein the dermal formulation comprises about 2% (w/w) compound of E2, about 71% (w/w) water, about 10% (w/w) glycerol, about 15% (w/w) propylene glycol, and about 2% (w/w) hydroxyethylcellulose.

E14. The dermal formulation according to any one of E4-E13, wherein the dermal formulation is a cream or a gel.

E15. The compound according to any one of E1-E3 for use as a medicament.

E16. A method of treating skin fibrosis comprising administering a therapeutically effective amount of a compound or a pharmaceutically acceptable salt thereof according to any one of E1-E3 or a dermal formulation of E4-E14 to a patient in need thereof.

E17. A method of treating skin fibrosis according to E16, wherein the skin fibrosis is caused by a disease or skin inflammation, a natural ageing process, scarring caused by burn injuries, by surgery, by plastic surgery, or by other skin injuries, acne, or keloids.

E18. A method for preparing (R)-3-(4-fluoro-2-hydroxyphenyl)-4-methyl-4,5-dihydro-1H-pyrazole-1-carboximidamide or a pharmaceutically acceptable salt thereof;
the method comprising:
   a) providing the compound 1-(2-(benzyloxy)-4-fluorophenyl)-2-((dimethylamino)methyl)prop-2-en-1-one;

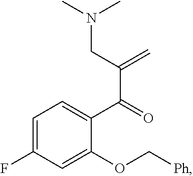 [SMILES: C=C(CN(C)C)C(=O)c1ccc(F)cc1OCc1ccccc1]

b) subjecting the compound 1-(2-(benzyloxy)-4-fluorophenyl)-2-((dimethylamino)methyl)prop-2-en-1-one to a ring closure process with aminoguanidine or aminoguanidine hydrochloride to obtain the compound 3-(2-(benzyloxy)-4-fluorophenyl)-4-((dimethylamino)methyl)-4,5-dihydro-1H-pyrazole-1-carboximidamide, optionally as a bis-trifluoroacetic acid salt,

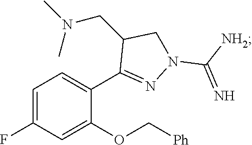 [SMILES: CN(C)CC1CN(C(=N)N)N=C1c1ccc(F)cc1OCc1ccccc1]

c) subjecting the compound 3-(2-(benzyloxy)-4-fluorophenyl)-4-((dimethylamino)methyl)-4,5-dihydro-1H-pyrazole-1-carboximidamide bis-trifluoroacetic acid salt to an elimination process to obtain the compound 3-(2-(benzyloxy)-4-fluorophenyl)-4-methylene-4,5-dihydro-1H-pyrazole-1-carboximidamide, optionally as a salt with an acid selected from CF₃CO₂H and MeS(O)₂OH

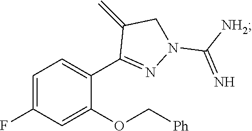 [SMILES: C=C1CN(C(=N)N)N=C1c1ccc(F)cc1OCc1ccccc1]

d) subjecting the compound 3-(2-(benzyloxy)-4-fluorophenyl)-4-methylene-4,5-dihydro-1H-pyrazole-1-carboximidamide, optionally as a salt with an acid selected from CF₃CO₂H and MeS(O)₂OH, to enantioselective hydrogenation to obtain the compound (R)-3-(2-(benzyloxy)-4-fluorophenyl)-4-methyl-4,5-dihydro-1H-pyrazole-1-carboximidamide, optionally as a salt with an acid selected from CF₃CO₂H and MeS(O)₂OH

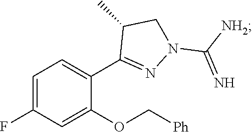 [SMILES: C[C@@H]1CN(C(=N)N)N=C1c1ccc(F)cc1OCc1ccccc1]

e) subjecting the compound (R)-3-(2-(benzyloxy)-4-fluorophenyl)-4-methyl-4,5-dihydro-1H-pyrazole-1-carboximidamide to deprotection, and optionally forming a pharmaceutically acceptable salt of the deprotected compound to obtain the compound (R)-3-(4-fluoro-2-hydroxyphenyl)-4-methyl-4,5-dihydro-1H-pyrazole-1-carboximidamide or a pharmaceutically acceptable salt thereof.

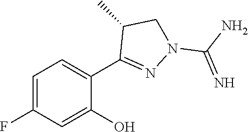 [SMILES: C[C@@H]1CN(C(=N)N)N=C1c1ccc(F)cc1O]

E19. The method according to E18, further comprising:
f) subjecting the compound (R)-3-(4-fluoro-2-hydroxyphenyl)-4-methyl-4,5-dihydro-1H-pyrazole-1-carboximidamide obtained in e) to diastereomeric salt formation with di-O,O′-p-toloyl-D-tartaric acid, followed by salt exchange to obtain the compound (R)-3-(4-fluoro-2-hydroxyphenyl)-4-methyl-4,5-dihydro-1H-pyrazole-1-carboximidamide with an increased enantiomeric purity, or a pharmaceutically acceptable salt thereof.

E20. The method according to any one of E18-E19, wherein the obtained compound (R)-3-(4-fluoro-2-hydroxyphenyl)-4-methyl-4,5-dihydro-1H-pyrazole-1-carboximidamide or a pharmaceutically acceptable salt thereof has an enantiomeric purity of at least about 80%, such as at least about 90%, such as at least about 95% or such as at least about 97%.

E21. A compound selected from:
1-(2-(Benzyloxy)-4-fluorophenyl)-2-((dimethylamino)methyl)prop-2-en-1-one;
3-(2-(Benzyloxy)-4-fluorophenyl)-4-((dimethylamino)methyl)-4,5-dihydro-1H-pyrazole-1-carboximidamide;
3-(2-(Benzyloxy)-4-fluorophenyl)-4-((dimethylamino)methyl)-4,5-dihydro-1H-pyrazole-1-carboximidamide bis-trifluoroacetic acid salt;
3-(2-(Benzyloxy)-4-fluorophenyl)-4-methylene-4,5-dihydro-1H-pyrazole-1-carboximidamide;
3-(2-(Benzyloxy)-4-fluorophenyl)-4-methylene-4,5-dihydro-1H-pyrazole-1-carboximidamide trifluoroacetic acid salt;
3-(2-(Benzyloxy)-4-fluorophenyl)-4-methylene-4,5-dihydro-1H-pyrazole-1-carboximidamide methanesulfonic acid salt;
(R)-3-(2-(Benzyloxy)-4-fluorophenyl)-4-methyl-4,5-dihydro-1H-pyrazole-1-carboximidamide;
(R)-3-(2-(Benzyloxy)-4-fluorophenyl)-4-methyl-4,5-dihydro-1H-pyrazole-1-carboximidamide methanesulfonic acid salt;
(R)-3-(4-fluoro-2-hydroxyphenyl)-4-methyl-4,5-dihydro-1H-pyrazole-1-carboximidamide; and
(R)-3-(4-fluoro-2-hydroxyphenyl)-4-methyl-4,5-dihydro-1H-pyrazole-1-carboximidamide di-O,O′-p-toloyl-D-tartaric acid salt.

E22. A compound of E21 for use in preparation of a compound or pharmaceutically acceptable salt according to any of E1-E3.

### EXAMPLES

### Example 1

### 3-(4-Fluoro-2-hydroxyphenyl)-4-methyl-4,5-dihydro-1H-pyrazole-1-carboximidamide hydrochloride

The racemic HCl salt was prepared by methods analogous to methods described in WO2016/207231A1.

¹H-NMR ((CD₃)₂SO) d 1.16 (d, 3H), 3.69 (dd, 1H), 4.04-4.17 (m, 2H), 6.77 (dt, 1H), 6.88 (dd, 1H), 7.76 (dd, 1H), 7.89 (broad s, 4H), 10.61 (broad s, 1H).

### Example 2

### (4R)-3-(4-Fluoro-2-hydroxyphenyl)-4-methyl-4,5-dihydro-1H-pyrazole-1-carboximidamide (Compound 2)

The enantiomers of the racemic 3-(4-fluoro-2-hydroxyphenyl)-4-methyl-4,5-dihydro-1H-pyrazole-1-carboximidamide were separated by chiral supercritical fluid chromatography (SFC).

Conditions: Lux A1 (21.2 mm×250 mm, 5 μm, column Temperature 40° C., flow Rate 50 mL/min, 100 BarG, isocratic conditions 30:70 MeOH:CO₂ (0.2% v/v NH₃).

3-(4-Fluoro-2-hydroxyphenyl)-4-methyl-4,5-dihydro-1H-pyrazole-1-carboximidamide hydrochlorid (5.94 g) was dissolved to 40 mg/mL in MeOH:CH₂Cl₂ (1:1) and purified by SFC. The eluates were evaporated, providing the R- and S-enantiomers, respectively, in base forms, both with ee (enantiomeric excess) 100.

¹H-NMR (CD₃OD) d 1.24 (d, 3H), 3.58 (dd, 1H), 3.97 (t, 1H), 4.19 (m, 1H), 6.19 (dt, 1H), 6.38 (dd, 1H), 7.33 (t, 1H).

### Example 3

### (4R)-3-(4-Fluoro-2-hydroxyphenyl)-4-methyl-4,5-dihydro-1H-pyrazole-1-carboximidamide hydrochloride (Compound 3)

A solution of HCl in MeOH (sat.) was added to a solution of (4R)-3-(4-fluoro-2-hydroxyphenyl)-4-methyl-4,5-dihydro-1H-pyrazole-1-carboximidamide in MeOH. The solution was then concentrated at reduced pressure and the residue was dried in vacuum at 40° C. to give the title compound (40% from racemic HCl salt, ee 100).

NMR as for racemate.

X-ray diffraction analysis (XRD) was used to determine the absolute configuration (4R). Data were collected at 100 K at Diamond Light Source, Didcot, England (λ=0.7000 Å), equipped with a Pilatus 6M-F detector. The structure (at 0.75 Å resolution) was solved using SHELXS2 and refined using SHELXL2 in combination with the graphical user interface SHELXLE3.

### Example 4

### Solubility of Compound 1 and Compound 2 in Water

The aq. solubility was determined by the addition of an excess of solid compound to MO-water. The solution and solid were mixed for 24 h at room temperature to ensure equilibrium. The excess solid compound was removed by filtration. The filtrate was then diluted and quantified with LC-UV against a 3-point calibration curve of the actual compound.
Compound 1 aq. solubility: 0.9 mg/mL
Compound 2 aq. solubility: 20.1 mg/mL

The results show a much better solubility (>20×) of the enantiomeric Compound 2, compared with the racemic Compound 1 in MQ-water after 24 h of equilibration.

### Example 5

### Solubility and Stability of Compound 2 and Compound 3 in Excipient Solvents, Including Water

Excess of each substance were added to the solvents. The samples were then stirred for 1 hour and left at room temperature overnight in the dark. The samples were centrifuged, and supernatants were transferred to vials and the compound concentrations were analysed by HPLC.

The samples were then stored in the dark at ambient temperature and analysed after one day, and after two and four weeks of storage to assess the stability of the compound solutions, i.e. detection of related impurities.

The solubilities (mg/mL) of Compound 2 in the following solvents were:
10.5 in HPLC water; 4.21 in EtOH; 36.5 in propylene glycol; >63* in PEG-400; 0.21 in caprylic/capric triglyceride; >15* in glycerol; 1.9 in DMI; >51* in transcutol

The solubilities (mg/mL) of Compound 3 in the following solvents were: 33.8 in HPLC water; 21.5 in EtOH; >51* in propylene glycol; 16.5 in PEG-400; 0.01 in caprylic/capric triglyceride; >34* in glycerol; 0.05 in DMI; 10.3 in transcutol.

(* all material dissolved)

The stability was not investigated in caprylic/capric triglycerides and in dimethyl isosorbide (DMI) as the solubilities were low, and not in transcutol since degradation was seen already at the initial analysis at 24 h for both compounds (2.91% for Compound 2 and 0.27% for Compound 3).

The detected related impurities (%) in Compound 2 solutions were at 24 h/2 weeks/4 weeks in
HPLC water: 0.13/0.54/0.84
EtOH: 0.24/0.54/0.69
Propylene glycol: 0.16/0.23/0.30
PEG-400: 0.18/0.39/0.50
Glycerol: 0.30/0.57/0.75

The detected related impurities (%) in Compound 3 solutions were at 24 h/2 weeks/4 weeks in
HPLC water: 0.10/0.08/0.08
EtOH: 0.10/0.07/0.08
Propylene glycol: 0.10/0.08/0.09
PEG-400: 0.10/0.08/0.09
Glycerol: 0.13/0.12/0.13

The results show that a slow but steady degradation is seen in all Compound 2 solutions, while all the Compound 3 solutions are stable during four weeks of storage.

### Example 6

### Stability and Hydrolysis of the Racemic HBr Salt Example 50 in WO2016/207231A1 and Compound 3 in Water

Each compound was dissolved in water (100 mg in 5 mL) and analysed by HPLC for purity and detection of any trace of urea impurities. The samples were then stored at 80° C., 90° C., and 100° C. and analysed by HPLC after 1, 3, 7, and 14 days of storage. For samples stored at 90° C. and 100° C. for 14 days, the samples were diluted with MeOH (10 mL) before analysis to dissolve any precipitated impurities.

Analytical HPLC system: Waters X-Bridge with C18 column (5 μm, 4.6 mm×150 mm)
Mobile phase: 0.1% TFA in water/acetonitrile gradient (95/5 to 5/95 during 14 min)
Automatic peak detection at 220 nm and integration.
Rₜ (parent compound): 7.8 min
Rₜ (urea derivative): 9.0 min

Results (% parent/% urea impurity) are expressed as peak % of total peak area.

Both compounds were pure (100/0) at day 0 and day 1 under all conditions. Degradation and urea formation was detected in the racemic HBr samples at 80 ° C. day 7 (99.7/0.3) and day 14 (99.4/0.4), at 90° C. day 7 (97.4/0.8) and day 14 (96.9/2.4), and at 100° C. day 3 (99.2/0.8), day 7 (91.4/5.2) and day 14 (49.7/28.4).

Degradation and urea formation was detected in the R-enantiomeric HCl samples at 80° C. day 14 (99.8/0.2), at 90° C. day 3 (99.8/0.2), day 7 (97.8/0.4) and day 14 (99.2/0.7), and at 100° C. day 3 (99.3/0.7), day 7 (96.5/1.2) and day 14 (99.3/0.7).

Some irregularities were noted concerning the last data points for the enantiomeric HCl salt; however, the overall results clearly show the enantiomeric HCl salt to be more stable under forced conditions, indicating a higher long-term stability.

The same stability experiments were performed using the racemic HCl salt as control and to assess a possible stereo-chemical effect, This compound was not 100% pure, showing 98.2% parent and 0.6% urea, but remained relatively stable at all conditions with 0.5-0.7% urea content and 96.2-99.4% of the parent compound. This demonstrates the high stability of the HCl salt and also rules out any stereo-chemical effect on stability.

### Example 7

### In Vitro Permeation Tests (IVPT) of Compound 2 and Compound 3 in Solvent Formulations (Donor Vehicle).

Dermatomed skin membranes from pig inner ears were used as membranes in the Bronaugh diffusion cell equipment. Compounds were dissolved (3% w/w) in the donor vehicle consisting of 50% water, 25% PEG-400 and 25% propylene glycol. Prior to the experiment the solubilities of Compound 2 (7.5% w/w) and Compound 3 (7.1% w/w) were determined by HPLC. Also, the pH of the formulations (pH 10.1 for Compound 2 formulation and pH 5.5 for Compound 3 formulation) was measured prior to application. The receptor solution consisted of a PBS buffer solution (pH 7.4). The donor solution was applied in an infinite dose (150 mg) with occlusion and sampling timepoints, to measure cumulative permeation, were 6, 12, 18, and 24 h.

At 24 h the membranes were cleaned, and the epidermis and dermis separated. The compounds were extracted with a water/acetonitrile mixture (50:50) and analyzed by HPLC.

### Results:

The cumulative permeation (μg/cm², compound concentration in receptor solution measured by HPLC) were:
Compound 2-0.00 (6 h), 0.08 (12 h), 0.22 (18 h), 0.53 (24 h)
Compound 3-0.02 (6 h), 0.05 (12 h), 0.17 (18 h), 0.38 (24 h)

### Amount of Compounds in the Skin Membranes

Compound 2 in epidermis: 991 μg/g, 7.9 μg/cm²
Compound 2 in dermis: 82.5 μg/g, 4.7 μg/cm²
Compound 3 in epidermis: 961 μg/g, 7.7 μg/cm²
Compound 3 in dermis: 64.7 μg/g, 4.0 μg/cm²

The results show that the permeation and accumulation in the tissue were similar for both compounds and no significant difference could be observed. After 24 h, >95% of the compound amounts remained in the skin tissue, while <5% permeated to the receptor solution.

The lower pH of the formulation with Compound 3 (pH 5.5) is better for skin applications. In addition, while the formulation of Compound 3 was stable, a relatively large degradation peak was found in the formulation of Compound 2, also previously detected in aqueous solutions in the stability study.

### Example 8

Compound 3 (2% w/w) Aqueous Cream Formulation (53.65% Water). Batch Size 100 q.

Aqueous phase: Polysorbate 60 (0.97 g) was heated to 50° C. and mixed with propylene glycol (15.00 g), glycerol (10.00 g), water (53.65 g) and Compound 3 (2.00 g) and stirred at 70° C. to a homogenous solution.

Lipid phase: A mixture of sorbitane monostearate (1.29 g), cetyl palmitate (1.93 g), cetostearyl alcohol (6.45 g) and 2-octyl-1 dodecanol (Kollicream OD) (8.71 g) was stirred at 70° C. to a homogenous solution.

The lipid phase was slowly added to the polar phase at 70° C. while stirring intensively. The mixture was then homogenised at 70° C. to give a homogenous emulsion, which was cooled to room temperature with moderate stirring.

### Example 9

Compound 3 (2% w/w) Aqueous Gel Formulations. Batch Size 25 g.

A mixture of EDTA (0.05% w/w), benzalkonium chloride (0.005% w/w) and water (81.745% w/w) was stirred at 60° C., then Carbopol 974 P (0.90% w/w) was added during vigorous stirring at 60° C. The mixture was cooled and stirred at room temperature for 45 minutes. Sodium hydroxide (0.30% w/w) was added, and the mixture was stirred for 30 minutes.

Compound 3 (2.00% w/w) was mixed with propylene glycol (15.00% w/w) and added to the gel mixture, which was stirred and mixed to a homogenous formulation.

### Example 10

Compound 3 (2% w/w) Aqueous Gel Formulation (96% Water). Batch Size 100 q.

Compound 3 (2.00 g) was dissolved in water (96.00 g). Hydroxyethylcellulose (Natrosol™, 250 HX Pharm, 2.00 g) was added and the mixture was stirred to a homogenous formulation.

### Example 11

Compound 3 (2% w/w) Aqueous Gel Formulation (71% Water). Batch Size 100 q.

Compound 3 (2.00 g) was dissolved in a mixture of water (71.00 g), glycerol (10.00 g) and propylene glycol (15.00 g). Hydroxyethylcellulose (Natrosol™, 250 HX Pharm, 2.00 g) was added and the mixture was stirred to a homogenous formulation.

### Example 12

### In Vitro Permeation Tests (IVPT) of Compound 3 in Cream and Gel Formulations

The experiments were performed as described in Example 7 using the 2% cream formulation (Example 8) and the 2% gel formulation (Example 11). Each formulation was applied once at a finite dose of 10 mg/cell (16 mg/cm²) with no occlusion. The pH values of the formulations were 4.39 (cream) and 5.98 (gel).

### Results:

The cumulative permeation (μg/cm²) to the receptor solution were:
For 2% Cream: 0.01 (6 h), 0.04 (12 h), 0.07 (18 h), 0.20 (24 h)
For 2% Gel: 0.00 (6 h), 0.01 (12 h), 0.01 (18 h), 0.03 (24 h)

### Amount of Compound 3 in the Skin Membranes

For 2% Cream in epidermis: 2586 μg/g, 20.7 μg/cm² (6.2% of dose)
For 2% Cream in dermis: 130 μg/g, 8.1 μg/cm² (2.4% of dose)
For 2% Gel in epidermis: 1443 μg/g, 11.5 μg/cm² (3.2% of dose)
For 2% Gel in dermis: 46 μg/g, 3.1 μg/cm² (0.9% of dose)

The results show that Compound 3 is effectively delivered to the skin from both the cream and gel formulations with high delivered dose fractions at 24 h, >8% and >4%, respectively. The results also show that the permeation of Compound 3 through the skin is very low as can be seen by the minute amounts accumulated in the reception solutions.

Together, this implies that the formulations are well suited for dermal topical delivery of Compound 3.

### Example 13

### Stability Studies of Compound 3 Cream and Gel Formulations

The stabilities of the 2% cream formulation of Example 8 and the 2% gel formulation of Example 11 were examined. Visual appearance, Compound 3 identity (HPLC) and assay (HPLC, % w/w), formation of related impurities (HPLC, peak area % of total), as well as pH, were determined at 0, 1, 2, 3, 6, and 9 months of storage at 25° C., 30° C., and 40° C.

### Results for the Cream Formulation:

Appearance—All samples “light grey cream”. Some phase separation detected at 1, 2, and 3 months at 40° C. No trends.
Identity—All samples positive.
Assay—Initial value 2.03%. All samples 1.98-2.14%. No trends.
Sum of related impurities—Initial value 0.10%. All samples 0.07-0.11%, except 0.15%, 0.14%, and 0.16% at 3, 6, and 9 m/40° C., respectively. pH—Initial value pH 4.39. All samples pH 4.36-4.52, except pH 3.84 at 9 m/40° C. The lower pH may be due to fatty acid formation from the lipid excipients in the cream.

### Results for the Gel Formulation:

Appearance—All samples “grey cream”, except for “grey, yellowish” at 9 m/25, 30, and 40° C.
Identity—All samples positive.
Assay: Initial value 2.01%. All samples 1.97-2.04%. No trends.
Sum of related impurities—Initial value 0.11%. All samples at 25° C. showed 0.10-0.11%. Samples at 30° C. showed 0.12% (1 m), 0.11% (2 m), 0.13% (3 m), 0.14% (6 m), 0.17% (9 m) and samples at 40° C. showed 0.14% (1 m), 0.18% (2 m), 0.27% (3 m), 0.34% (6 m), 0.53% (9 m).
pH—Initial value pH 5.98. All samples pH 5.91-6.12. No trends.

The results demonstrate that the formulations are generally very stable at 25° C. The only obvious stability related changes observed over nine months of storage were the discolouration of the gel formulation at 9 m (from very slightly yellow at 25° C. to slightly yellow/brown at 40° C.) and the small but clear formation of impurities in the gel formulation stored at 40° C.

### Example 14

### 1-(2-(Benzyloxy)-4-fluorophenyl)ethan-1-one

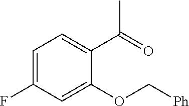 [SMILES: CC(=O)c1ccc(F)cc1OCc1ccccc1]

A mixture of 4′-fluoro-2′-hydroxyacetophenone (46.2 g, 0.30 mol), benzyl bromide (51.3 g, 0.30 mol), and K₂CO₃ (81.2 g, 0.60 mol) in MeCN (250 mL) was stirred at reflux temperature (b.p. 82° C.) for 2 h. After cooling, the reaction mixture was filtered and the filtrate was concentrated at reduced pressure to give the title compound as a white solid (72.0 g, 98%).

¹H-NMR (CD₃OD) δ 2.52 (s, 3H), 5.22 (s, 2H), 6.77 (dt, 1H), 7.01 (dd, 1H), 7.35 (t, 1H), 7.41 (t, 2H), 7.50 (d, 2H), 7.78 (dd, 1H).

### Example 15

### 1-(2-(Benzyloxy)-4-fluorophenyl)-2-((dimethylamino)methyl)prop-2-en-1-one

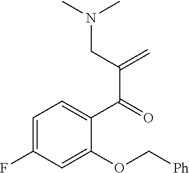 [SMILES: C=C(CN(C)C)C(=O)c1ccc(F)cc1OCc1ccccc1]

### 1,3-Dioxolane as formaldehyde equivalent

A mixture of 1-(2-(benzyloxy)-4-fluorophenyl)ethan-1-one (14.7 g, 60.2 mmol), dimethylamine hydrochloride (49.1 g, 602 mmol) and conc HCl (37%, 0.6 mL) in 1,3-dioxolane (250 mL) was stirred at 86° C. overnight. After cooling, the reaction mixture was partitioned between water (250 mL) and methyl tert-butyl ether (200 mL). After phase separation, water (50 mL) and K₂CO₃ (75 g) were added to the aqueous phase followed by extraction with methyl tert-butyl ether (2×350 mL). The organic phase was washed with water, dried (Na₂SO₄), filtered, and concentrated at reduced pressure to give the title compound as an oil (18.0 g, 95%).

¹H-NMR (CDCl₃) δ 2.18 (s, 6H), 3.23 (s, 2H), 5.05 (s, 2H), 5.80 (s, 1H), 6.00 (d, 1H), 6.67-6.74 (m, 2H), 7.29-7.45 (m, 6H).

### Paraformaldehyde as Formaldehyde Equivalent:

A mixture of 1-(2-(benzyloxy)-4-fluorophenyl)ethan-1-one (4.0 g, 16.4 mmol), dimethylamine hydrochloride (5.3 g, 64 mmol), paraformaldehyde (2.14 g, 64 mmol) and AcOH (8.0 mL) in iso-PrOAc (20 mL) was stirred under N₂ (g) at 90° C. overnight. After cooling, the reaction mixture was concentrated at reduced pressure, diluted with methyl tert-butyl ether (40 mL) and basified (pH about 10) by addition of aq 2M K₂CO₃ (60 mL). The organic phase was separated and washed with water, dried (Na₂SO₄), filtered, and concentrated at reduced pressure to give the title compound as an oil (4.75 g, 93%).

### Example 16

### 3-(2-(Benzyloxy)-4-fluorophenyl)-4-((dimethylamino)methyl)-4,5-dihydro-1H-pyrazole-1-carboximidamide bis-trifluoroacetic acid salt

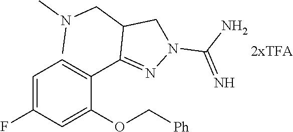 [SMILES: CN(C)CC1CN(C(=N)N)N=C1c1ccc(F)cc1OCc1ccccc1]

An aq. solution of NaOH (5M, 11.1 mL) was added to a solution of aminoguanidine hydrochloride (6.19 g, 55 mmol) in water (33 mL) and EtOH (100 mL) at 0° C. The combined solutions were added to 1-(2-(benzyloxy)-4-fluorophenyl)-2-((dimethylamino)methyl)prop-2-en-1-one (13.3 g, 42.4 mmol) and the mixture was stirred at room temperature overnight and then partitioned between EtOAc and brine. The organic phase was separated, dried (Na₂SO₄) and concentrated at reduced pressure. The residue, containing the base form of the product (ca 19 g), was dissolved in EtOAc (100 mL) and trifluoroacetic acid (6.34 mL, 83 mmol) was added. Petroleum ether (100 mL) was slowly added to precipitate the product. The mixture was stirred for 5 h and the product was collected by filtration, washed with EtOAc and dried to give the title compound (11.9 g, 47%) as a white solid.

¹H-NMR (CD₃OD) δ 2.59 (broad s, 6H), 3.19 (dd, 1H), 3.28-3.35 (m, 1H, solvent at 3.31 ppm), 4.11 (dd, 1H), 4.25 (t, 1H), 4.53 (m, 1H), 5.24 (dd, 1H), 5.29 (dd, 1H), 6.86 (dt, 1H), 7.14 (dd, 1H), 7.38-7.47 (m, 3H), 7.54 (d 2H), 7.91 (dd, 1H).

### Example 17

### 3-(2-(Benzyloxy)-4-fluorophenyl)-4-methylene-4,5-dihydro-1H-pyrazole-1-carboximidamide trifluoroacetic acid salt

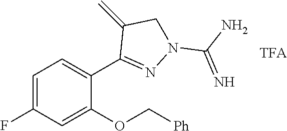 [SMILES: C=C1CN(C(=N)N)N=C1c1ccc(F)cc1OCc1ccccc1]

Oxone ((KHSO₅)₂·KHSO₄·K₂SO₄, 3.33 g, 5.42 mmol) was added to a mixture of 3-(2-(benzyloxy)-4-fluorophenyl)-4-((dimethylamino)methyl)-4,5-dihydro-1H-pyrazole-1-carboximidamide bis-trifluoroacetic acid salt (4.99 g, 8.86 mmol) and Na₂HPO₄(H₂O)₂ (6.57 g, 36.9 mmol) in EtOAc (80 mL) and water (20 mL). After the addition, the slurry dissolved, and the mixture was stirred vigorously at room temperature for 20 min giving a two-phase system. Na₂SO₄ (26.7 g) was added and stirring was continued for 10 min. The clear organic phase (containing the N-oxide) was separated, and the aqueous/solid residue was extracted with EtOAc (2×20 mL). The combined organic phases were concentrated to a volume of about 80 mL for azeotropic removal of water. The solution was stirred at 60° C. for 50 min. After cooling, trifluoroacetic acid (0.4 mL) was added to the stirred solution, followed by addition of petroleum ether (40 mL) and water (40 mL) to give a white precipitate. After stirring vigorously for 5 min, the solids were collected by filtration, washed with water and EtOAc/petroleum ether (1:1), and dried to give the title compound as white crystals (3.59 g, 96%).

¹H-NMR (CD₃OD) δ 4.75 (t, 2H), 5.13 (s, 2H), 5.33 (t, 1H), 5.57 (t, 1H), 6.84 (dt, 1H), 7.06 (dd, 1H), 7.29-7.44 (m, 6H).

### Example 18

### 3-(2-(Benzyloxy)-4-fluorophenyl)-4-methylene-4,5-dihydro-1H-pyrazole-1-carboximidamide

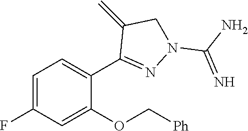 [SMILES: C=C1CN(C(=N)N)N=C1c1ccc(F)cc1OCc1ccccc1]

1 M aq. NaOH (9.0 mL, 9.0 mmol) was added to a solution of 3-(2-(benzyloxy)-4-fluorophenyl)-4-methylene-4,5-dihydro-1H-pyrazole-1-carboximidamide trifluoroacetic acid salt (3.59 g, 8.5 mmol) in MeOH (36 mL). The solution was then cooled and stirred on an ice bath. Water (18 mL) was added to precipitate the product, which was collected by filtration after stirring for 10 min. The product was washed with MeOH/water (1:3) and water and dried to give the title compound (2.24 g, 81%).

¹H-NMR (CD₃OD) δ 4.65 (t, 2H), 5.08 (t, 1H), 5.11 (s, 2H), 5.34 (t, 1H), 6.79 (dt, 1H), 6.99 (dd, 1H), 7.27-7.41 (m, 6H).

### Example 19

### 3-(2-(Benzyloxy)-4-fluorophenyl)-4-methylene-4,5-dihydro-1H-pyrazole-1-carboximidamide methanesulfonic acid salt

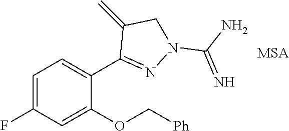

A mixture of (3-(2-(benzyloxy)-4-fluorophenyl)-4-methylene-4,5-dihydro-1H-pyrazole-1-carboximidamide trifluoroacetic acid salt (35.0 g, 79.9 mmol) and methanesulfonic acid (15.4 g, 160 mmol) in MeOH (350 mL) and EtOH (800 mL) was stirred for 10 minutes at room temperature. Solvents were evaporated at reduced pressure to a volume of approx. 300 mL followed by addition of petroleum ether (1000 mL) to precipitate the product MSA salt, which was collected by filtration, washed with petroleum ether and dried to give the title compound (30.8 g, 92%).

¹H-NMR (CD₃OD) δ 2.70 (s, 3H), 4.75 (t, 2H), 5.14 (s, 2H), 5.33 (t, 1H), 5.58 (t, 1H), 6.84 (dt, 1H), 7.06 (dd, 1H), 7.27-7.47 (m, 6H).

### Example 20

### (S)-3-(2-(Benzyloxy)-4-fluorophenyl)-4-methyl-4,5-dihydro-1H-pyrazole-1-carboximidamide

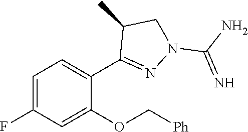 [SMILES: C[C@H]1CN(C(=N)N)N=C1c1ccc(F)cc1OCc1ccccc1]

0.04% eq Rh(nbd)₂/SL-J212-1 (CAS RN® 849924-41-0) (ratio: 1/1.2) as catalyst.

A mixture of 3-(2-(benzyloxy)-4-fluorophenyl)-4-methylene-4,5-dihydro-1H-pyrazole-1-carboximidamide (1 eq, 0.12 M), methane sulfonic acid (2.6 eq) and the catalyst in THF/MeOH (9:1) was stirred under hydrogen gas (15 bar) overnight. Chiral HPLC analysis showed full conversion and an enantiomeric R/S ratio of 7:93.

### Example 21

### (R)-3-(2-(Benzyloxy)-4-fluorophenyl)-4-methyl-4,5-dihydro-1H-pyrazole-1-carboximidamide

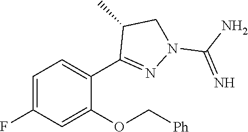 [SMILES: C[C@@H]1CN(C(=N)N)N=C1c1ccc(F)cc1OCc1ccccc1]

0.02% eq Rh(nbd)₂/SL-J212-2 (CAS RN® 849924-42-1) (ratio: 1/1.2, 0.32 mM) as catalyst was prepared under argon in THF at 40° C. for 15 min. The catalyst solution was added to a solution of 3-(2-(benzyloxy)-4-fluorophenyl)-4-methylene-4,5-dihydro-1H-pyrazole-1-carboximidamide methanesulfonic acid salt (10.09 g, 24.0 mmol) and methane sulfonic acid (3.69 g, 38.4 mmol) in THF/MeOH (9:1, 200 mL) under argon. The reaction mixture was then stirred under hydrogen gas (15 bar) at 30° C. for 27 h. Chiral HPLC analysis showed full conversion and an enantiomeric R/S ratio of 94:6. The reaction mixture was concentrated at reduced pressure to give 14.9 g of crude residue. The residue (10.05 g) was partitioned in i-PrOAc (200 mL) and 2 M aq K₂CO₃ (125 mL) and the organic phase was then concentrated at reduced pressure to give the crude title compound (6.10 g, quant. yield)

¹H-NMR ((CD₃)₂SO) δ 1.01 (d, 3H), 3.42 (dd, 1H), 3.73 (m, 1H), 3.87 (t, 1H), 5.20 (s, 2H), 6.30 (bras s, 2H), 6.86 (dt, 1H), 7.15 (dd, 1H), 7.36 (t, 1H), 7.42 (t, 2H), 7.48 (d, 2H), 7.73 (dd, 1H).

### Example 22

### (R)-3-(4-Fluoro-2-hydroxyphenyl)-4-methyl-4,5-dihydro-1H-pyrazole-1-carboximidamide

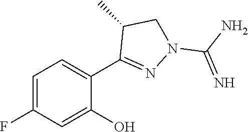 [SMILES: C[C@@H]1CN(C(=N)N)N=C1c1ccc(F)cc1O]

A mixture of (R)-3-(2-(benzyloxy)-4-fluorophenyl)-4-methyl-4,5-dihydro-1H-pyrazole-1-carboximidamide (490 mg, 1.50 mmol) and Pd/C (10%, 49 mg) in MeOH (10 mL) was stirred under H₂ (g) at room temperature for 4 h. After filtration, the filtrate was concentrated to dryness to give the crude title compound (370 mg, quant. yield).

¹H-NMR (CD₃)₂SO) δ 1.12 (d, 3H), 3.53 (dd, 1H), 3.86 (t, 1H), 4.38 (m, 1H), 5.82 (dt, 1H), 5.92 (dd, 1H), 7.51 (t, 1H).

### Example 23

### (R)-3-(4-Fluoro-2-hydroxyphenyl)-4-methyl-4,5-dihydro-1H-pyrazole-1-carboximidamide hydrochloride

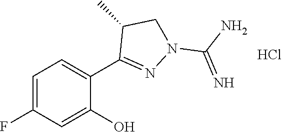 [SMILES: C[C@@H]1CN(C(=N)N)N=C1c1ccc(F)cc1O ; Cl]

HCl (aq, 37%, 0.143 mL) was added to a solution of (R)-3-(4-fluoro-2-hydroxyphenyl)-4-methyl-4,5-dihydro-1H-pyrazole-1-carboximidamide (370 mg, 1.57 mmol) in EtOH (5.5 mL). The solution was concentrated at reduced pressure to 1.5 mL and then i-PrOAc (7.0 mL) was added. After stirring for 30 min the precipitated HCl-salt was collected by filtration, washed with i-PrOAc (2×2 mL) and dried to give the title compound (240 mg, 56%).

¹H-NMR ((CD₃)₂SO) δ 1.16 (d, 3H), 3.68 (dd, 1H), 4.04-4.16 (m, 2H), 6.78 (dt, 1H), 6.88 (dd, 1H), 7.76 (dd, 1H), 7.89 (broad s, 4H), 10.59 (s, 1H).

Chiral HPLC: R/S ratio 96.25:3.75

### Example 24

### (R)-3-(4-fluoro-2-hydroxyphenyl)-4-methyl-4,5-dihydro-1H-pyrazole-1-carboximidamide D-DpTTA salt

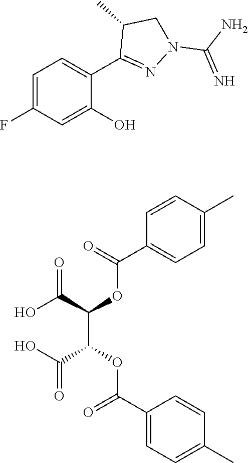 [SMILES: C[C@@H]1CN(C(=N)N)N=C1c1ccc(F)cc1O ; Cc1ccc(C(=O)O[C@H](C(=O)O)[C@H](OC(=O)c2ccc(C)cc2)C(=O)O)cc1]

A solution of di-O,O′-para-toloyl-D-tartaric acid (656 mg, 1.69 mmol) in acetone (6 mL) was added to a solution of (R)-3-(4-fluoro-2-hydroxyphenyl)-4-methyl-4,5-dihydro-1H-pyrazole-1-carboximidamide (400 mg, 1.69 mmol) in acetone (46 mL). After stirring for 20 h, the precipitate was collected by filtration, washed with acetone (2×2 mL) and dried to give the title product (810 mg, 77%).

¹H-NMR ((CD₃)₂SO) δ 1.12 (d, 3H), 2.35 (s, 6H), 3.57 (dd, 1H), 3.98 (t, 1H), 4.05 (m, 1H), 5.64 (s, 2H), 6.76 (dt, 1H), 6.81 (dd, 1H), 7.31 (d, 4H), 7.72 (dd, 1H), 7.85 (d, 4H), 8.19 (very broad s), 10.75 (broad s, 1H).

### Example 25

### (R)-3-(4-Fluoro-2-hydroxyphenyl)-4-methyl-4,5-dihydro-1H-pyrazole-1-carboximidamide hydrochloride

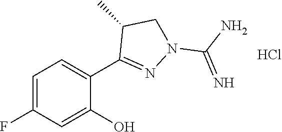 [SMILES: C[C@@H]1CN(C(=N)N)N=C1c1ccc(F)cc1O ; Cl]

Aq HCl (37%, 0.075 mL) was added to a slurry of (R)-3-(4-fluoro-2-hydroxyphenyl)-4-methyl-4,5-dihydro-1H-pyrazole-1-carboximidamide D-DpTTA salt (500 mg, 0.80 mmol, Example 24) in abs. EtOH (2.5 mL) to give a clear solution. i-PrOAc (7.5 mL) was added, and the solution was stirred for 1 h. The precipitate was collected by filtration, washed with i-PrOAc and dried to give the title product (157 mg, 72%).

¹H-NMR ((CD₃)₂SO) δ 1.16 (d, 3H), 3.68 (dd, 1H), 4.07 (t, 1H), 4.10 (m, 1H), 6.78 (dt, 1H), 6.88 (dd, 1H), 7.76 (dd, 1H), 7.88 (broad s, 4H), 10.57 (s, 1H). Chiral HPLC: R/S ratio 99.99:0.01

### Example 26

### (R)-3-(4-fluoro-2-hydroxyphenyl)-4-methyl-4,5-dihydro-1H-pyrazole-1-carboximidamide D-DpTTA salt

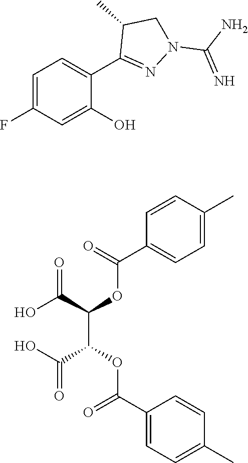 [SMILES: C[C@@H]1CN(C(=N)N)N=C1c1ccc(F)cc1O ; Cc1ccc(C(=O)O[C@H](C(=O)O)[C@H](OC(=O)c2ccc(C)cc2)C(=O)O)cc1]

### Chiral Purification by Trituration

A sample of (R)-3-(4-fluoro-2-hydroxyphenyl)-4-methyl-4,5-dihydro-1H-pyrazole-1-carboximidamide D-DpTTA salt (0.5 g, R/S ratio 92:8) was triturated in EtOH (10 mL) and i-PrOAc (5 mL) at 50° C. overnight. After cooling, the salt was collected by filtration to give a product with increased enantiomeric purity (0.31 g, 62%, R/S ratio 98.7:1.3).

### Example 27

### (R)-3-(4-fluoro-2-hydroxyphenyl)-4-methyl-4,5-dihydro-1H-pyrazole-1-carboximidamide hydrochloride

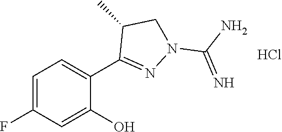 [SMILES: C[C@@H]1CN(C(=N)N)N=C1c1ccc(F)cc1O ; Cl]

Salt exchange in large scale.

Aq. conc. HCl (3.06 kg) was added to a mixture of (R)-3-(4-fluoro-2-hydroxyphenyl)-4-methyl-4,5-dihydro-1H-pyrazole-1-carboximidamide di-O,O′-para-toloyl-D-tartaric acid salt (17.5 kg, mol) in EtOH (265 L) and the solution was stirred for 1 h to form a clear solution. The solution was concentrated to approx. 90 L The HCl salt was precipitated by addition of i-PrOAc (265 L). After stirring for 1 h at room temperature, and then at 5° C. overnight, the product was collected by filtration, washed with i-PrOAc and dried to give the title compound (6.70 kg, 87%).

¹H-NMR ((CD₃)₂SO) δ 1.16 (d, 3H), 3.69 (dd, 1H), 4.04-4.17 (m, 2H), 6.77 (dt, 1H), 6.88 (dd, 1H), 7.76 (dd, 1H), 7.89 (broad s, 4H), 10.61 (broad s, 1H).

1. A compound (4R)-3-(4-fluoro-2-hydroxyphenyl)-4-methyl-4,5-dihydro-1H-pyrazole-1-carboximidamide hydrochloride.

2. The compound according to claim 1, wherein the compound has an enantiomeric purity of at least 90%.

3. A dermal formulation comprising the compound according to claim 1 and one or more pharmaceutically acceptable excipients or carriers.

4. The dermal formulation according to claim 3, wherein the dermal formulation is an aqueous formulation.

5. The dermal formulation according to claim 4, wherein the dermal formulation comprises ≥20% (w/w) of water.

6. The dermal formulation according to claim 5, wherein the dermal formulation comprises 50-98% (w/w) of water.

7. The dermal formulation according to claim 4, wherein the dermal formulation further comprises 1-30% (w/w) propylene glycol.

8. The dermal formulation according to claim 4, wherein the dermal formulation further comprises 1-20% (w/w) glycerol.

9. The dermal formulation according to claim 4, wherein the dermal formulation comprises 0.1-2% (w/w) of (4R)-3-(4-fluoro-2-hydroxyphenyl)-4-methyl-4,5-dihydro-1H-pyrazole-1-carboximidamide hydrochloride.

10. The dermal formulation according to claim 4, wherein the dermal formulation is a cream or a gel.

11. A method of treating skin fibrosis comprising administering a therapeutically effective amount of the compound according to claim 1 to a patient in need thereof.

12. The method of treating skin fibrosis according to claim 11, wherein the skin fibrosis is caused by a disease or skin inflammation, a natural ageing process, scarring caused by burn injuries, by surgery, by plastic surgery, or by other skin injuries, acne, or keloids.

13-16. (canceled)

17. A compound selected from the group consisting of:
3-(2-(Benzyloxy)-4-fluorophenyl)-4-methylene-4,5-dihydro-1H-pyrazole-1-carboximidamide;
3-(2-(Benzyloxy)-4-fluorophenyl)-4-methylene-4,5-dihydro-1H-pyrazole-1-carboximidamide trifluoroacetic acid salt;
3-(2-(Benzyloxy)-4-fluorophenyl)-4-methylene-4,5-dihydro-1H-pyrazole-1-carboximidamide methanesulfonic acid salt;
(R)-3-(2-(Benzyloxy)-4-fluorophenyl)-4-methyl-4,5-dihydro-1H-pyrazole-1-carboximidamide; and
(R)-3-(4-fluoro-2-hydroxyphenyl)-4-methyl-4,5-dihydro-1H-pyrazole-1-carboximidamide.

18-19. (canceled)

20. A compound
(R)-3-(4-fluoro-2-hydroxyphenyl)-4-methyl-4,5-dihydro-1H-pyrazole-1-carboximidamide di-O,O′-p-toloyl-D-tartaric acid salt.
